(19) United States
(12) Patent Application Publication
(10) Pub. No.: US 2023/0271996 A1
(43) Pub. Date: Aug. 31, 2023

(54) PROCESS FOR PREPARING CEDAZURIDINE

(71) Applicant: ScinoPharm Taiwan, Ltd., Shan-Hua (TW)

(72) Inventors: Phyllis Wang, Shan-Hua (TW); Kuan-Hsun Huang, Shan-Hua (TW); Tsung-Cheng Hu, Shan-Hua (TW)

(21) Appl. No.: 18/064,975

(22) Filed: Dec. 13, 2022

Related U.S. Application Data
(60) Provisional application No. 63/293,765, filed on Dec. 25, 2021.

(51) Int. Cl. C07H 19/06; C07H 1/06; B01J 31/02

(52) U.S. Cl. CPC C07H 19/06; B01J 31/0237; C07H 1/06; B01J 2231/4288; B01J 2531/002

(57) ABSTRACT

A one-pot process for preparing cedazuridine of formula (I),

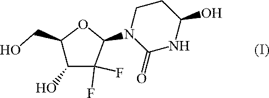 [SMILES: O=C1N[C@H](O)CCN1[C@@H]1O[C@H](CO)[C@@H](O)C1(F)F]

comprising: subjecting a compound of formula (M3)

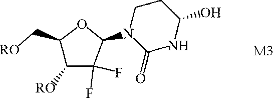

to deprotection and then epimerization in a reactor in the presence of a catalyst to obtain a reaction mixture comprising cedazuridine, wherein R on the compound of formula (M3) is independently selected from the group consisting of Ac (acetyl), Bz (benzoyl), p-nitrobenzoyl and OtBu (tert-butyloxycarbonyl), and the deprotection and epimerization are conducted in the same reactor without isolating after the deprotection and before the epimerization; and isolating cedazuridine from the reaction mixture.

### CROSS-REFERENCES TO RELATED APPLICATIONS

This application claims priority to U.S. Provisional Application No. 63/293,765 filed Dec. 25, 2021, which is incorporated in its entirety for all purposes.

### FIELD

This disclosure relates to a process of making cedazuridine.

### BACKGROUND OF THE INVENTION

Cedazuridine and decitabine, sold under the brand name Inqovi®, is a fixed-dose combination medication for the treatment of adults with myelodysplastic syndromes (MDS) and chronic myelomonocytic leukemia (CMML). Decitabine/cedazuridine was approved for medical use in the United States and in Canada in July 2020.

Cedazuridine is represented by formula (I):

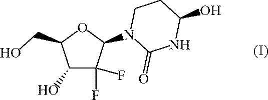 [SMILES: O=C1N[C@H](O)CCN1[C@@H]1O[C@H](CO)[C@@H](O)C1(F)F]

Cedazuridine has the chemical name of “(4R)-2′-deoxy-2ʹ,2′-difluoro-3,4,5,6-tetrahydrouridine” or “(4R)-1-[(2R,4R,5R)-3,3-difluoro-4-hydroxy-5-(hydroxymethyl)oxolan-2-yl]-4-hydroxy-1,3-diazinan-2-one.” Cedazuridine has a molecular formula of C₉H₁₄F₂N₂O₅ and a molecular weight of 268.21 Da.

The synthesis of cedazuridine has been a challenging and costly task due to multiple functional groups present in the chemical structure. There have been several synthetic methods reported in the relevant literatures for the synthesis of cedazuridine.

U.S. Pat. No. 9,567,363 B2 and J. Med. Chem. 2014, 57, 2582-2588 disclose the preparation of cedazuridine as shown in Scheme 1. Cedazuridine can be prepared from converting gemcitabine through hydrogenation and hydrolysis of gemcitabine followed by reduction under NaBH₄ in MeOH. Then the crude cedazuridine is purified by preparative HPLC. The total yield is about 18%.

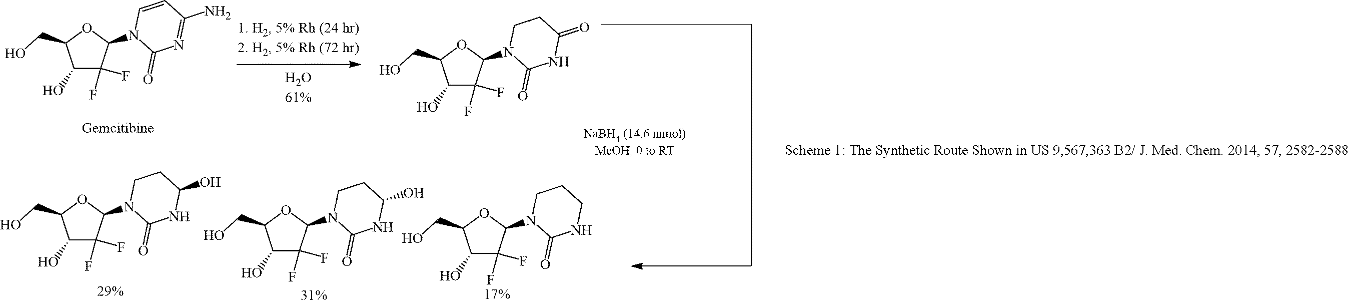

U.S. Pat. No. 9,834,576 B2 discloses an alternative route for the preparation of cedazuridine as shown in Scheme 2. It also involves hydrolysis and hydrogenation of gemcitabine intermediate and then follows by the reduction under NaBH₄ in DCM/EtOH with CeCl₃.7H₂O/ as adduct. Subsequently, deprotection of the Bz group with NH₃/MeOH and epimerization by DBU/CH₃CN leads to crude cedazuridine.. After the purification, the yield of cedazuridine is 73-86.1%

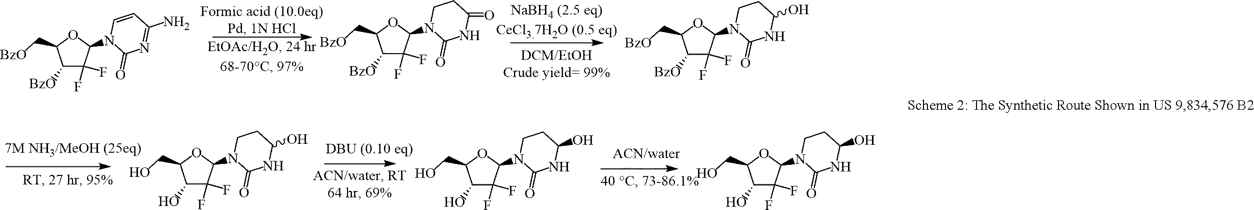

WO2021071890A1 has demonstrated a similar route of preparing cedazuridine, as shown in Scheme 3. WO2021071890A1 also involves hydrolysis and hydrogenation of gemcitabine intermediate and followed by reduction under NaBH₄ in DCM/EtOH with CeCl₃.7H₂O/ as adduct. The improvement deprotection in WO2021071890A1 is a work-up of the deprotected compound under non-aqueous conditions and simplification of the isolation procedure. Later, before recrystallization of crude cedazuridine, the mixture should be performed with epimerization by DBU with ACN_{(aq)} as a solvent to give crude cedazuridine. Then, crystallization by acetone/water can lead to cedazuridine with 61% yield. The total yield of five steps of cedazuridine synthesis is 28%.

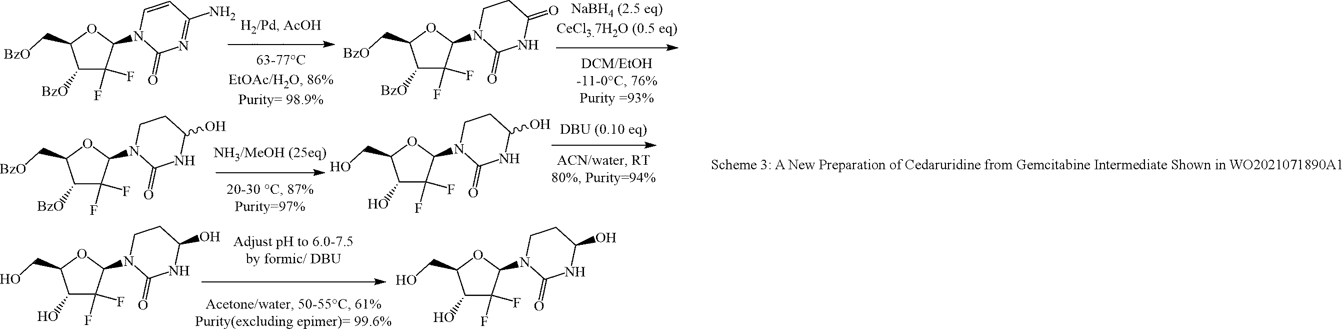

Despite the above-described processes, there remains a need for the development of improved processes for the preparation of cedazuridine. The present disclosure addresses this need and provides related advantages as well.

### SUMMARY OF THE INVENTION

The present invention provides a one-pot process for preparing cedazuridine, represented by formula (I):

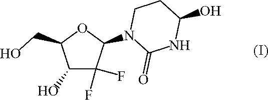 [SMILES: O=C1N[C@H](O)CCN1[C@@H]1O[C@H](CO)[C@@H](O)C1(F)F]

comprising: subjecting a compound of formula (M3)

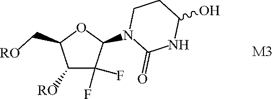

to deprotection and then epimerization in a reactor in the presence of a catalyst to obtain a reaction mixture comprising cedazuridine, wherein R on the compound of formula (M3) is independently selected from the group consisting of Ac (acetyl), Bz (benzoyl), p-nitrobenzoyl and OtBu (tert-butyloxycarbonyl), and the deprotection and epimerization are conducted in the same reactor without isolating after the deprotection and before the epimerization; and isolating cedazuridine from the reaction mixture.

Preferably, R on the compound of formula (M3) is Bz (benzoyl).

The isolating may comprise crystallizing the cedazuridine from the reaction mixture. The crystallizing is preferably conducted in the presence of a cedazuridine seed.

The catalyst may be selected from the group consisting of 1,1,3,3-tetramethylguanidine (TMG), 1,5,7-triazabicyclo(4.4.0)dec-5-ene (TBD), 7-methyl-1,5,7-triazabicyclo(4.4.0)dec-5-ene (MTBD), 2-tert-butyl-1,1,3,3-tetramethylguanidine (Barton’s base), and combinations thereof, more preferably 1,1,3,3-Tetramethylguanidine (TMG).

According to a preferred embodiment, the process comprises controlling cedazuridine to have a particle size with D90 equal to or less than 100 µm, preferably equal to or less than 60 µm, more preferably equal to or less than 20 µm.

According to a preferred embodiment, the process comprises a further purifying step, which may comprise: 1) adding a solvent to the crude cedazuridine; 2) performing slurry the reduced cedazuridine in the solvent to obtain a purified cedazuridine. The purifying step is preferably conducted after the above-discussed controlling the particle size of cedazuridine. The solvent is preferably selected from the group consisting of acetone, THF, MeCN, water, and combinations thereof. In another preferred embodiment, the solvent is a co-solvent system of acetone and water.

In another aspect, the present invention provides a process for preparing cedazuridine of formula (I),

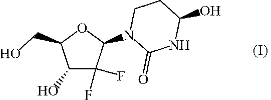 [SMILES: O=C1N[C@H](O)CCN1[C@@H]1O[C@H](CO)[C@@H](O)C1(F)F]

comprising:
a) reducing particle size with D90 of crude cedazuridine to no greater 100 µm;
b) mixing the reduced cedazuridine obtained in step a) with a solvent to obtain a mixture comprising cedazuridine; and
c) isolating cedazuridine from the mixture of step b) to obtain the purified cedazuridine

Preferably, the isolating comprises performing slurry the reduced cedazuridine in the solvent to obtain the purified cedazuridine.

The particle size of D90 of step b) may be controlled to equal to or less than 100 µm, preferably equal to or less than 60 µm, and equal to or less than 20 µm.

The solvent of step c) may be selected from the group consisting of acetone, THF, MeCN, water, and combinations thereof. As a preferred embodiment, the solvent may be a co-solvent system of acetone and water.

Preferably, prior to the step of controlling the particle size of cedazuridine, the process comprises:
subjecting a compound of formula (M3)

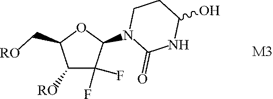

to deprotection and then epimerization in a reactor in the presence of a catalyst to obtain a reaction mixture comprising cedazuridine, wherein R on the compound of formula (M3) is independently selected from the group consisting of Ac (acetyl), Bz (benzoyl), p-nitrobenzoyl and OtBu (tert-butyloxycarbonyl), and the deprotection and epimerization are conducted in the same reactor without isolating after the deprotection and before the epimerization; and
isolating the crude cedazuridine from the reaction mixture.

### DETAILED DESCRIPTION OF PREFERRED EMBODIMENTS OF THE INVENTION

### Examples

The following examples are provided to illustrate, but not to limit, the present invention.

The synthetic route for the preparation of cedazuridine is described below:

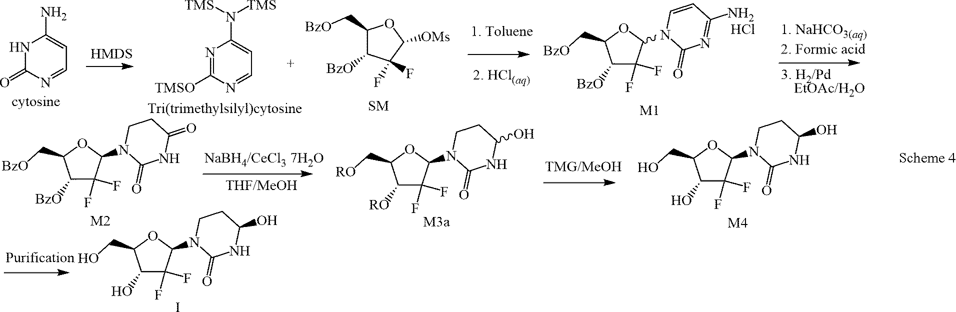

### Example 1: Preparation of 3′,5′-Di-O-benzoyl-2′-Deoxy-2′,2′-difluoro-1,3-diazinan-2,4-dione (M2)

EtOAc (596 mL), 5%NaHCO₃ (197 mL) and M1 (3′,5′-Di-O-benzoyl-2′-deoxy-2′,2′-difluorocytidine hydrochloride) (39.73 g) were added to the hydrogenator. Formic acid (14.8 mL) and Pd/C (1.59 g) were added to the hydrogenator. The reaction mixture was stirred under pressure of H₂ at 60-70° C. for overnight. The mixture was filtered to remove Pd/C and washed with EtOAc (198 mL). The water layer was removed from filtrate by phase separation. The organic layer was washed with 5% NaHCO₃ and water. The organic layer was concentrated by reducing pressure and recrystallized by adding n-heptane (358 mL). The solid was filtrated and dried to obtain M2 (37.11 g) in 87% yield with 99.55% purity.

### Example 2: Preparation of 3′,5′-Di-O-benzoyl-2′-Deoxy-2′,2′-difluoro-4-hydroxy-1,3-diazinan-2-one (M3a)

CeCl₃.7H₂O (70.68 g) and M2 (90 g, 189.7 mmol) were dissolved in MeOH (360 mL) and THF (540 mL). NaBH₄ (12.92 g) was added to the mixture and stirred for 5 hours. The resulting mixture was then quenched with acetone (90 mL). The mixture was washed with brine and 5% NaHCO₃ and then extracted with EtOAc (450 mL). The organic layer was separated, and extracted aqueous layer by EtOAc (450 mL). Combined organic layers were washed with water and then the solvent-swap was carried out with EtOAc. The EtOAc layer was concentrated by reducing pressure and crystallized by adding n-heptane (1350 mL). The solid was filtrated and dried to give M3a (74.42 g) in 82.34% yield with 98.71% purity.

### Example 3: One-pot Reaction for the Preparation of Crude Cedazuridine

M3a (10.00 g, 20.92 mmol) and TMG (0.132 g, 1.05 mmol) were charged into a suitable reactor and dissolved in MeOH (300 mL). The mixture was stirred at 15° C. for 18 hours. The resulting mixture was concentrated and then MeCN (170 mL) was added to the mixture. Then the solution mixture was concentrated and the water (5.0 mL) and cedazuridine seed were added to the mixture. The mixture was concentrated and stirred at 10-20° C. for 1 hour, then the solvent-swap was carried out with MeCN. The solution was filtrated and dried to obtain dried product (4.92 g) in 83% yield. The dried product was further micronized to give crude cedazuridine particles having a D90 equal to or less 100 µm.

### Example 4: Purification of Crude Cedazuridine

The crude cedazuridine (10.00 g, 37.28 mmol, D90=117 µm) was added with co-solvent of acetone (13 mL, 3.2 vol) and water (6 mL, 0.6 vol) at room temperature. The slurry mixture was heated to 40° C. then cooled to RT for two cycles to give cedazuridine with 0.92% alpha-epimer with purity 99.92% (excluding alpha-epimer).

The crude cedazuridine (10.00 g, 37.28 mmol, D90=60 µm) was added with co-solvent of acetone (13 mL, 3.2 vol) and water (6 mL, 0.6 vol) at room temperature. The slurry mixture was heated to 40° C. then cooled to RT for two cycles to give Cedazuridine with 0.63% alpha-epimer with purity 99.94% (excluding alpha-epimer).

The crude cedazuridine (10.00 g, 37.28 mmol, D90=11.3 µm) was added with co-solvent of acetone (13 mL, 3.2 vol) and water (6 mL, 0.6 vol) at room temperature. The slurry mixture was heated to 40° C. then cooled to RT for two cycles to give cedazuridine with 0.21% alpha-epimer with purity 99.96% (excluding alpha-epimer).

TABLE 1

| | US 9,834,576 B2 | | WO2021071890A1 | | Embodiment of Present invention |
|---|---|---|---|---|---|
| Deprotection | 95% | Total:65.6% | 87% | Total: 69.6% | 83% |
| Epimerization | 69% | | 80% | | |
| Purification (Cedazuridine/Alpha-epimer) | 86.1 (98.6/1.4) | | 61% (99.6/0.4) | | 81% (99.79/0.21) |
| Yield from M3 to Cedazuridine | 56% | | 42% | | 67% |

As can be seen in Table 1 above, the present invention provides an improved process in comparison with prior art documents. The present invention can not only reduce the reaction steps but also increase overall yield.

TABLE 2

| Before Purification | | D90 (µm) | Purification | |
|---|---|---|---|---|
| Alpha epimer | Purity (excluding alpha epimer) | | | |
| | | | alpha epimer | Purity (excluding alpha epimer) |
| 1.45% | 99.82% | 117 | 0.92% | 99.92% |
| | | 60 | 0.63% | 99.94% |
| | | 11 | 0.21% | 99.96% |
| Cedazuridine from RLD (INQOVI®)* | | NA | 0.3% | NA |

*RLD: reference listed drug which is an approved drug product to which new generic versions are compared to show that they are bioequivalent.

To meet the criteria of RLD, the present invention found that controlling the particle size (D90) of the crude cedazuridine below 100 µm can obtain pure cedazuridine having the alpha-epimer less than 1% as shown in Table 2.

What is claimed is:

1. A one-pot process for preparing cedazuridine of formula (I), 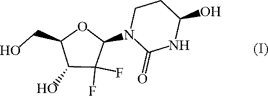 [SMILES: O=C1N[C@H](O)CCN1[C@@H]1O[C@H](CO)[C@@H](O)C1(F)F]
comprising:
subjecting a compound of formula (M3) 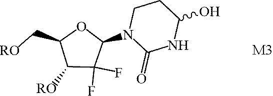
to deprotection and then epimerization in a reactor in the presence of a catalyst to obtain a reaction mixture comprising cedazuridine, wherein R on the compound of formula (M3) is independently selected from the group consisting of Ac (acetyl), Bz (benzoyl), p-nitrobenzoyl and OtBu (tert-butyloxycarbonyl), and the deprotection and epimerization are conducted in the same reactor without isolating after the deprotection and before the epimerization; and
isolating cedazuridine from the reaction mixture.

2. The process of claim 1 wherein R on the compound of formula (M3) is Bz (benzoyl).

3. The process of claim 1 wherein the isolating comprises crystallizing the cedazuridine from the reaction mixture.

4. The process of claim 3 wherein the crystallizing is conducted in the presence of a cedazuridine seed.

5. The process of claim 1, wherein the catalyst is selected from the group consisting of 1,1,3,3-tetramethylguanidine (TMG), 1,5,7-triazabicyclo(4.4.0)dec-5-ene (TBD), 7-methyl-1,5,7-triazabicyclo(4.4.0)dec-5-ene (MTBD), 2-tert-butyl-1,1,3,3-tetramethylguanidine (Barton’s base), and combinations thereof.

6. The process of claim 1, wherein the catalyst is 1,1,3,3-tetramethylguanidine (TMG).

7. The process of claim 1, wherein the process further comprises reducing particle size with D90 of the isolated cedazuridine to no greater than 100 µm.

8. The process of claim 7, wherein the particle size with D90 of the isolated cedazuridine is reduced to no greater than 60 µm.

9. The process of claim 7, wherein the particle size with D90 of the isolated cedazuridine is reduced to no greater than 20 µm.

10. The process of claim 7, comprising a further step of purifying which comprises: 1) mixing the reduced cedazuridine with a solvent; 2) performing slurry the reduced cedazuridine in the solvent to obtain a purified cedazuridine.

11. The process of claim 10, wherein the solvent is selected from the group consisting of acetone, THF, MeCN, water, and combinations thereof.

12. The process of claim 11, wherein the solvent is a co-solvent system of acetone and water.

13. A process for preparing a purified cedazuridine of formula (I), 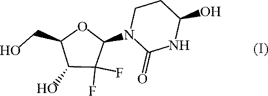 [SMILES: O=C1N[C@H](O)CCN1[C@@H]1O[C@H](CO)[C@@H](O)C1(F)F]
comprising:
d) reducing a particle size with D90 of crude cedazuridine to no greater 100 µm;
e) mixing the reduced cedazuridine obtained in step a) with a solvent to obtain a mixture comprising cedazuridine; and
f) isolating cedazuridine from the mixture of step b) to obtain the purified cedazuridine.

14. The process of claim 13 wherein the isolating comprises performing slurry the reduced cedazuridine in the solvent to obtain the purified cedazuridine.

15. The process of claim 13 wherein the particle size with D90 of the crude cedazuridine is reduced to no greater than 60 µm.

16. The process of claim 13 wherein the particle size with D90 of the crude cedazuridine is reduced to no greater than 20 µm.

17. The process of claim 13 comprising, prior to the step of reducing:
subjecting a compound of formula (M3) 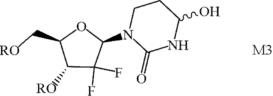
to deprotection and then epimerization in a reactor in the presence of a catalyst to obtain a reaction mixture comprising cedazuridine, wherein R on the compound of formula (M3) is independently selected from the group consisting of Ac (acetyl), Bz (benzoyl), p-nitrobenzoyl and OtBu (tert-butyloxycarbonyl), and the deprotection and epimerization are conducted in the same reactor without isolating after the deprotection and before the epimerization; and
isolating the crude cedazuridine from the reaction mixture.
